(19) United States
(12) Patent Application Publication
(10) Pub. No.: US 2025/0389560 A1
(43) Pub. Date: Dec. 25, 2025

(54) BIOLOGICAL SENSOR AND BIOLOGICAL INFORMATION DETECTING DEVICE

(71) Applicant: Murata Manufacturing Co., Ltd., Nagaokakyo-shi (JP)

(72) Inventor: Noriaki OKUDA, Nagaokakyo-shi (JP)

(21) Appl. No.: 19/311,882

(22) Filed: Aug. 27, 2025

Related U.S. Application Data
(63) Continuation of application No. PCT/JP2024/011243, filed on Mar. 22, 2024.

(30) Foreign Application Priority Data
Apr. 21, 2023 (JP) 2023-070251

(51) Int. Cl. G01D 11/24; G01D 11/28; H01F 5/00

(52) U.S. Cl. CPC G01D 11/245; G01D 11/28; H01F 5/00; H01F 2005/006

(57) ABSTRACT

A biological sensor includes a housing including a protuberant curved surface bulging in a first direction, and a substrate inside the housing and to be used with the first direction of the housing directed toward a living body. The substrate includes first and second portions at mutually different positions in the first direction and a jutting portion between the first and second portions such that the first portion protrudes farther in the first direction than the second portion. A biological detector is provided on a surface of the first portion orthogonal or substantially orthogonal to the first direction.

### CROSS REFERENCE TO RELATED APPLICATIONS

This application claims the benefit of priority to Japanese Patent Application No. 2023-070251 filed on Apr. 21, 2023 and is a Continuation Application of PCT Application No. PCT/JP2024/011243 filed on Mar. 22, 2024. The entire contents of each application are hereby incorporated herein by reference.

### BACKGROUND OF THE INVENTION

### 1. Field of the Invention

The present invention relates to biological sensors and biological information detecting devices each including a biological sensor.

### 2. Description of the Related Art

In a biological sensor including a light emitter and a light receiver, for example, Japanese Unexamined Patent Application Publication No. 2016-047105 discloses a biological sensor configured to detect a weak light component. A biological information detecting device using the biological sensor includes an entirely flat sensor substrate on which a light-emitting portion and a light-receiving portion are mounted.

In addition, a rigid flexible PCB (Printed Circuit Board) and a manufacturing method thereof are disclosed in, for example, Japanese Unexamined Patent Application Publication No. 2015-213169. The rigid flexible PCB includes a first rigid region, a second rigid region, and a flexible region connecting the first rigid region and the second rigid region.

In the biological information detecting device disclosed in Japanese Unexamined Patent Application Publication No. 2016-047105, since the sensor substrate on which the light-emitting portion and the light-receiving portion are mounted is entirely flat, the sensor substrate interferes with a curved surface region when the sensor substrate is relatively large, and the sensor substrate cannot thus be brought very close to a human body that is a detection object. As described above, when a large sensor substrate is used, and the interval between the sensor substrate and a human body is widened, the signal to noise ratio of a biological signal becomes worse. In addition, when the amount of emission light from a light-emitting portion is increased, the signal to noise ratio can be increased, but power consumption is increased.

### SUMMARY OF THE INVENTION

Example embodiments of the present invention provide biological sensors each including a substrate with a large area while a light emitter and a light receiver are brought close to a living body that is a detection object, and biological information detecting devices each including such biological sensors.

A biological sensor according to an example embodiment of the present invention includes a housing including a protuberant curved surface bulging in a first direction, and a substrate inside the housing, the biological sensor being used with the first direction of the housing directed toward a living body, in which the substrate includes a first portion and a second portion at mutually different positions in the first direction and a jutting portion extending between the first portion and the second portion such that the first portion protrudes farther in the first direction than the second portion. An opposite surface of the substrate from a surface on the jutting portion side is recessed. A biological detector is provided on a surface of the first portion orthogonal or substantially orthogonal to the first direction.

A biological information detecting device according to an example embodiment of the present invention includes a biological sensor according to an example embodiment of the present invention, and a processor configured or programmed to process detection information of the biological sensor.

Example embodiments of the present invention provide biological sensors each including a substrate with a large area while the light emitter and the light receiver are brought close to a living body that is a detection object, and biological information detecting devices including such biological sensors.

The above and other elements, features, steps, characteristics and advantages of the present invention will become more apparent from the following detailed description of the example embodiments with reference to the attached drawings.

### BRIEF DESCRIPTION OF THE DRAWINGS

FIG. 1 is a perspective view of a substrate defining a portion of a biological sensor according to a first example embodiment of the present invention.

FIG. 2 is a half sectional view of a biological sensor 100 configured by covering, with a housing 2, a substrate 1 illustrated in FIG. 1, a light emitter 31, and light receivers 32A and 32B.

FIG. 3 is a sectional view illustrating a state where the biological sensor 100 is used for a living body HB.

FIG. 4 is a block diagram illustrating an electric-circuit configuration of a biological information detecting device 101.

FIG. 5 is a half sectional view of a biological sensor 100 according to a second example embodiment of the present invention.

FIG. 6 is a partial enlarged sectional view of the biological sensor 100 according to the second example embodiment of the present invention.

FIG. 7A is a perspective view of a substrate 1 defining a portion of a biological sensor according to a third example embodiment. FIG. 7B is a sectional view taken along an X-Z plane at a position passing through the center of the substrate 1.

FIG. 8 is a perspective view of a substrate 1 defining a portion of a biological sensor 100 according to a fourth example embodiment of the present invention.

FIG. 9A is a sectional view of a housing 2 and the substrate 1 in a state of being separated from each other. FIG. 9B is a sectional view of the substrate 1 in a state of being combined with the housing 2 into one body.

FIG. 10 is a perspective view of a substrate 1 defining a portion of a biological sensor according to a fifth example embodiment of the present invention.

FIG. 11 is a sectional view illustrating the positional relationship between the substrate 1 and a housing 2.

FIG. 12A is a perspective view of a substrate 1 defining a portion of a biological sensor according to a sixth example embodiment. FIG. 12B is a sectional view taken along an X-Z plane at a position passing through the center of the substrate 1.

FIG. 13 is a perspective view of a substrate 1 according to a seventh example embodiment of the present invention.

FIG. 14 is a sectional view of a biological sensor 100 including the substrate 1 illustrated in FIG. 13 and a housing 2.

FIG. 15 is a perspective view of a substrate 1 according to an eighth example embodiment of the present invention.

FIG. 16 is a perspective view of a substrate 1 according to a ninth example embodiment of the present invention.

FIG. 17 is a sectional view of a biological sensor 100 including the substrate 1 illustrated in FIG. 16 and a housing 2.

FIG. 18 illustrates a state of a biological information detecting device 101 put around the left wrist of a human body.

FIG. 19A is a side view of a substrate 1 that is a portion of a biological sensor to be accommodated inside the biological information detecting device, and FIG. 19B is a perspective view of the substrate 1.

FIG. 20 is a side view of the biological information detecting device 101.

### DETAILED DESCRIPTION OF THE EXAMPLE EMBODIMENTS

A biological sensor according to an example embodiment of the present invention includes a housing including a protuberant curved surface bulging in a first direction, and a substrate inside the housing, the biological sensor being used with a first direction side of the housing directed toward a living body. The substrate includes a first portion and a second portion at mutually different positions in the first direction and a jutting portion between the first portion and the second portion such that the first portion protrudes farther in the first direction than the second portion. A light emitter and a light receiver are provided on a surface of the first portion in the first direction. The light emitter and the light receiver are configured to detect biological information.

A biological information detecting device according to an example embodiment of the present invention includes a biological sensor according to an example embodiment of the present invention, and a processor configured or programmed to process detection information of the biological sensor.

Hereinafter, example embodiments of the present invention will be described with reference to the drawings. The same reference signs are used for the same or corresponding portions in each drawing. In view of ease of description or understanding of the scope, separate example embodiments will be described for convenience of description, but configurations in different example embodiments may be partially replaced or combined. After a second example embodiment, description of common features with a first example embodiment will be omitted, and only differences will be described. In particular, similar actions and advantageous effects achieved by the same or similar configurations will not be described in every example embodiment.

### First Example Embodiment

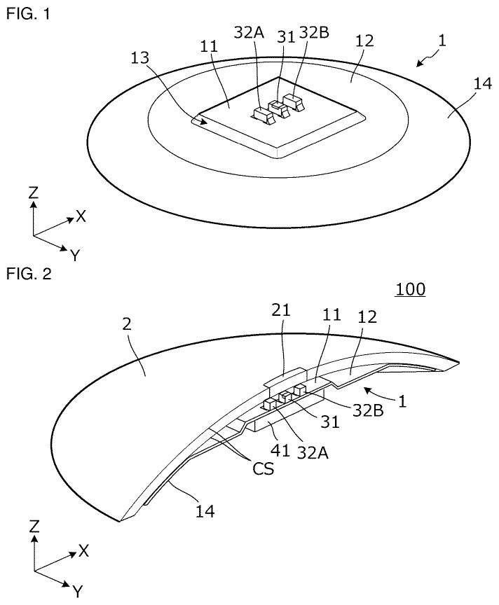

FIG. 1 is a perspective view of a substrate defining a portion of a biological sensor according to a first example embodiment of the present invention. As in FIG. 1, a substrate 1, and a light emitter 31 and light receivers 32A and 32B mounted on the substrate 1 are included. The substrate 1 has a circular or substantially circular shape when viewed in a Z direction. The Z direction corresponds to a “first direction”.

The substrate 1 includes a first portion 11 and a second portion 12 at mutually different positions in the Z direction and includes a jutting portion 13 between the first portion 11 and the second portion 12 such that the first portion 11 protrude farther in the first direction than the second portion 12. An opposite surface of the substrate (back surface) from the surface on the side of the jutting portion 13 is recessed. A curved surface portion 14 is provided around the second portion 12. In this example embodiment, an opposite surface (back surface) of the substrate from the surface on the side of the curved surface portion 14 is recessed.

The substrate 1 is, for example, a multilayer substrate including a liquid crystal polymer layer corresponding to a radio-frequency signal of about 5 GHz and a conductor pattern layer. The substrate 1 is shaped as in FIG. 1 by, for example, heating and pressing a flat plate-shaped multilayer substrate by using a mold. Here, since the substrate 1 includes multiple layers of a thermoplastic resin, plastic deformation through heat press using a mold is facilitated. Among thermoplastic resins, for example, a liquid crystal polymer with excellent radio-frequency characteristics may preferably be used.

The light emitter 31 and the light receivers 32A and 32B correspond to a “biological detector”. The light emitter 31 and the light receivers 32A and 32B are disposed on a surface of the first portion 11 orthogonal or substantially orthogonal (that is, an X-Y plane) to the Z direction. The light emitter 31 is, for example, an LED, a VCSEL (Vertical Cavity Surface Emitting Laser), a resonator LED, or the like, and the light receivers 32A and 32B are each, for example, a photodiode or a phototransistor.

FIG. 2 is a half sectional view of a biological sensor 100 configured by covering, with a housing 2, the substrate 1, the light emitter 31, and the light receivers 32A and 32B illustrated in FIG. 1. The example of FIG. 2 is illustrated as a sectional view taken along an X-Z plane at a position passing through the center of the biological sensor 100.

As illustrated in FIG. 2, the biological sensor 100 includes the housing 2 including a protuberant curved surface CS bulging in the Z direction and the substrate 1 disposed inside the housing 2. The housing 2 includes an optical window 21 at a position where the optical window 21 faces the light emitter 31 and the light receivers 32A and 32B. For example, a rectangular or substantially rectangular opening is provided in a portion of the housing 2, and the optical window 21 is fitted into the opening. The optical window 21 has a material transparency to the light emitted by the light emitter 31 and the light to be received by the light receivers 32A and 32B. On the other hand, a portion of the housing 2 other than the optical window 21 shields or absorbs the emission light of the light emitter 31 and the reception light of the light receivers 32A and 32B. Such a portion of the housing 2 other than the optical window 21 is, for example, a black resin in which a black resist composition is mixed into a resin.

The substrate 1 and the housing 2 are combined into one body by adhering the curved surface portion 14 of the substrate 1 and an inner surface of the curved surface CS of the housing 2 with an adhesive layer therebetween. The housing 2 may be configured by providing a black coverlay on a surface of a resin.

The opposite surface of the substrate 1, to a protruding direction of the first portion 11 is recessed, and a first component 41 is provided in the space of the recessed portion. Thus, the thickness space of the protruding portion of the first portion 11 of the substrate 1 is effectively used. That is, upsizing of the substrate 1 and the biological sensor as a whole due to the disposition of the first component 41 at a position on the opposite surface to the protruding direction of the first portion 11 is reduced or prevented. The first component is not limited to a simple electronic component alone such as a simple IC. The first component 41 may be mounted on at least a portion of the opposite surface of the substrate 1 to the protruding direction of the first portion 11.

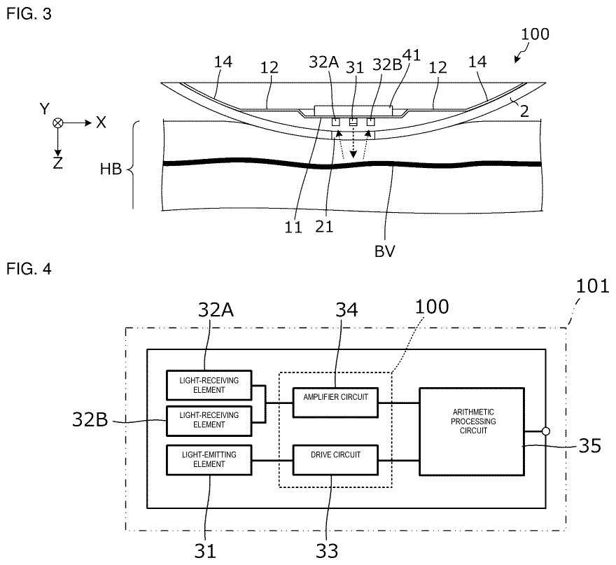

FIG. 3 is a sectional view illustrating a state where the biological sensor 100 is used for a living body HB. In FIG. 3, to make illustration of each portion clear, hatching illustration at the sectional position is omitted. As in the FIG. 3, the biological sensor 100 is used with the Z direction of the housing 2 directed toward a living body. The light applied from the light emitter 31 impinges on a blood vessel BV inside the living body HB, and a portion of the reflection light thereof is received by both or one of the light receivers 32A and 32B.

The housing 2 includes a protuberant curved surface bulging in the first direction such as a spherical surface or a hyperboloid surface of one sheet, but an opening portion of the housing 2 may be closed with a flat plate-shaped lid.

FIG. 4 is a block diagram illustrating an electric-circuit configuration of a biological information detecting device 101. The light emitter 31 is connected to a drive circuit 33. The drive circuit 33 dynamically drives the light emitter 31. The light receivers 32A and 32B are connected to an amplifier circuit 34. The amplifier circuit 34 amplifies the intensity of light reception signals provided by the light receivers 32A and 32B. An arithmetic processing circuit 35 is connected to the drive circuit 33 and the amplifier circuit 34. The arithmetic processing circuit 35 processes predetermined biological-sensor information such as pulse frequency and blood oxygen concentration, based on the drive process of the light emitter 31 performed by the drive circuit 33 and a light reception signal through output by the amplifier circuit 34, and outputs the information outside.

Characteristic portions of the biological sensor and the biological information detecting device in the first example embodiment are described as follows.

The substrate 1 is a three-dimensional multilayer substrate formed by three-dimensional deformation. The light emitter 31 and the light receivers 32A and 32B are mounted on the first portion 11 that is a protruding portion. The jutting portion 13 is provided around the first portion 11 and is a protruding portion. Thus, (a) integration of the entire or substantially the entire inside space of the housing 2 can be achieved. (b) The signal to noise ratio of a light signal can be increased because close distances from the light emitter 31 and the light receivers 32A and 32B to a living body can be achieved. (c) The rigidity of the first portion 11 can be increased with the jutting portion 13. In addition, since the area of the substrate 1 can be increased, a routable region and a component-mounting region can be increased.

In addition, since the substrate 1 includes the curved surface portion 14 extending along the curved surface of the housing 2, the rigidity of the entire substrate 1 including the curved surface portion 14 can be increased.

As described above, according to the present example embodiment, the substrate 1 having a large substrate area can be used while the light emitter 31 and the light receivers 32A and 32B are brought close to a detection object of a living body.

### Second Example Embodiment

In a second example embodiment of the present invention, an example of a biological sensor including a second component other than the first component in the first example embodiment will be described.

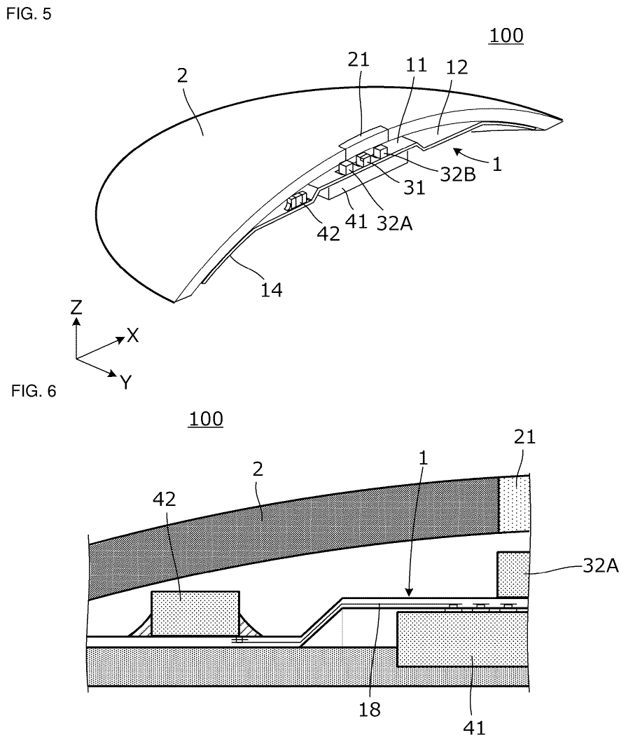

FIG. 5 is a half sectional view of a biological sensor 100 according to the second example embodiment. The example of FIG. 5 is illustrated as a sectional view taken along an X-Z plane at a position passing through the center of the biological sensor 100.

The structure of a housing 2 of the biological sensor 100 according to the second example embodiment is the same or substantially the same as the structure of the housing 2 in the first example embodiment illustrated in FIG. 2. A second component 42 is provided on a surface of a substrate 1 in the protruding direction of a first portion 11. That is, the biological sensor 100 according to the second example embodiment includes the second component 42 including at least a portion mounted on the surface of the substrate 1 in the protruding direction, and other configurations are the same as or similar to the configuration of the biological sensor 100 in the first example embodiment.

FIG. 6 is a partial enlarged sectional view of the biological sensor 100 according to the second example embodiment. FIG. 6 particularly illustrates the electric-circuit connection relationship between a first component 41 and the second component 42. In the present example embodiment, the substrate 1 is a multilayer substrate, and some terminals of the first component 41 that is an IC are electrically connected to terminals of the substrate 1. Similarly, a terminal of the second component 42 that is a chip component is electrically connected to a terminal of the substrate 1. Thus, a terminal of the first component 41 is connected to the second component 42 with an inner layer wire 18 inside the substrate 1 therebetween.

According to the present example embodiment, the thickness space of the protruding portion of the first portion 11 of the substrate 1 is effectively used. That is, upsizing of the substrate 1 and the biological sensor as a whole due to the second component 42 on the first portion 11 is reduced or prevented.

### Third Example Embodiment

In a third example embodiment of the present invention, an example including a substrate different in shape from the substrate included in the biological sensor 100 in the first and second example embodiments will be described.

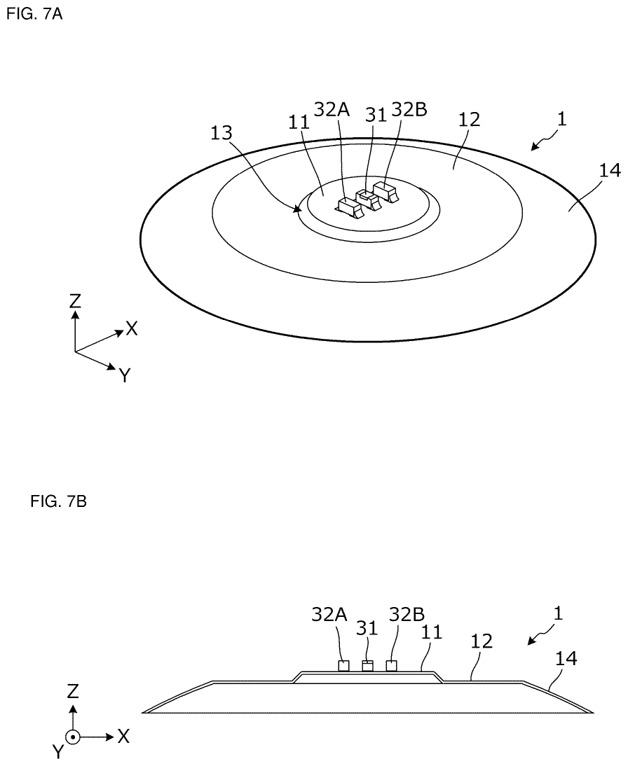

FIG. 7A is a perspective view of a substrate 1 defining a portion of a biological sensor according to the third example embodiment. FIG. 7B is a sectional view taken along an X-Z plane at a position passing through the center of the substrate 1. In FIG. 7B, to make illustration of each portion clear, hatching illustration at the sectional position of the substrate 1 is omitted. A light emitter 31 and light receivers 32A and 32B are mounted on the substrate 1. The substrate 1 has a circular or substantially circular outside shape when viewed in the Z direction. The substrate 1 includes a first portion 11 and a second portion 12 at mutually different positions in the Z direction and includes a jutting portion 13 between the first portion 11 and the second portion 12 such that the first portion 11 protrudes farther in the first direction than the second portion 12.

As is clear from a comparison with FIG. 1, the outside shape of the first portion 11 and the jutting portion 13 are circular or substantially circular when viewed in the Z direction. As described above, the first portion 11 may also be circular or substantially circular as with the second portion 12. In addition, for example, the second portion 12 may be rectangular or substantially rectangular, and a curved surface portion 14 may also be rectangular or substantially rectangular.

### Fourth Example Embodiment

In a fourth example embodiment of the present invention, an example including a substrate different in shape from the substrate included in the biological sensor in the third example embodiment will be described.

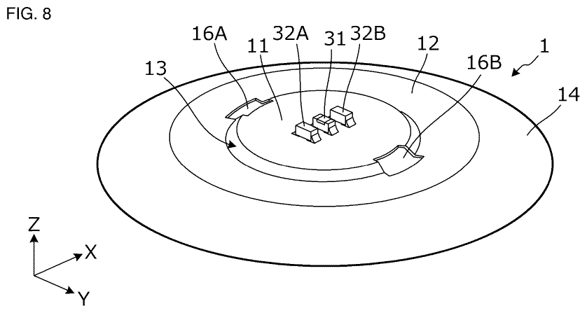

FIG. 8 is a perspective view of a substrate 1 defining a portion of a biological sensor 100 according to the fourth example embodiment. In the present example embodiment, a jutting portion 13 of the substrate 1 includes opening portions 16A and 16B.

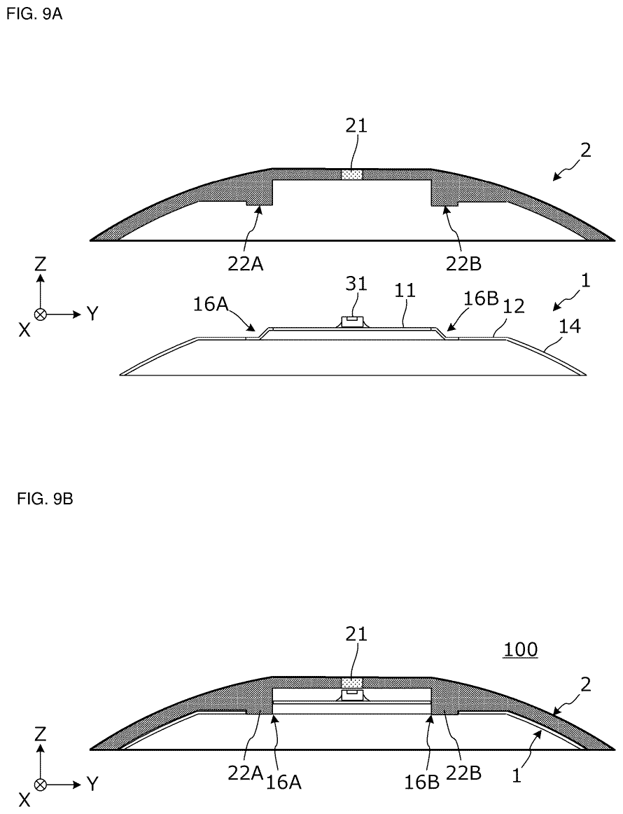

FIG. 9A is a sectional view of a housing 2 and the substrate 1 in a state of being separated from each other. FIG. 9B is a sectional view of the substrate 1 in a state of being combined with the housing 2 into one body. The housing 2 includes engagement portions 22A and 22B. The engagement portions 22A and 22B are engaged with the opening portions 16A and 16B provided in the substrate, respectively, and the housing 2 and the substrate 1 are thus combined into one body.

According to the present example embodiment, there is no need to adhere a curved surface portion 14 of the substrate 1 and an inner surface-side curved surface of the housing 2 with an adhesive layer therebetween, and weight reduction as a whole can be achieved. In addition, rotation positions of the substrate 1 and the housing 2 in a rotational direction can be positioned with the engagement portions 22A and 22B of the housing 2 and the opening portions 16A and 16B of the substrate. Thus, stable optical characteristics can be obtained even when an optical window 21 is rectangular or substantially rectangular.

### Fifth Example Embodiment

In a fifth example embodiment of the present invention, an example including a first portion different in shape from the first portions of the substrates in the above-described example embodiments will be described.

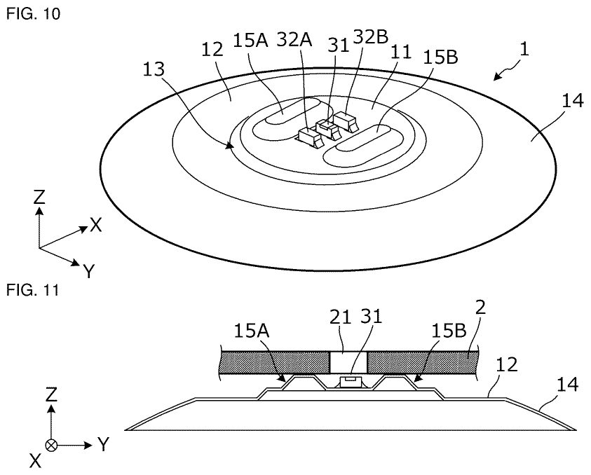

FIG. 10 is a perspective view of a substrate 1 defining a portion of a biological sensor according to the fifth example embodiment. FIG. 11 is a sectional view illustrating the positional relationship between the substrate 1 and a housing 2. In the present example embodiment, projecting body-shaped portions 15A and 15B are provided on a first portion 11 of the substrate 1. Although a light emitter 31 and light receivers 32A and 32B are mounted on the first portion 11, the protruding heights of the projecting body-shaped portions 15A and 15B are as high as the light emitter 31 and the light receivers 32A and 32B or are high enough not to allow the light emitter 31 and the light receivers 32A and 32B to be in contact with the housing 2.

According to the present example embodiment, a gap between each of the light emitter 31, the light receiver 32A, and the light receiver 32B, and an optical window 21 of the housing can be ensured. In addition, the light emitter 31 and the light receivers 32A and 32B are not in direct contact with the housing 2, and mechanical stress that is applied to the light emitter 31 and the light receivers 32A and 32B can be prevented.

### Sixth Example Embodiment

In a sixth example embodiment of the present invention, an example including a first portion different in shape from the first portions of the substrates in the above-described example embodiments will be described.

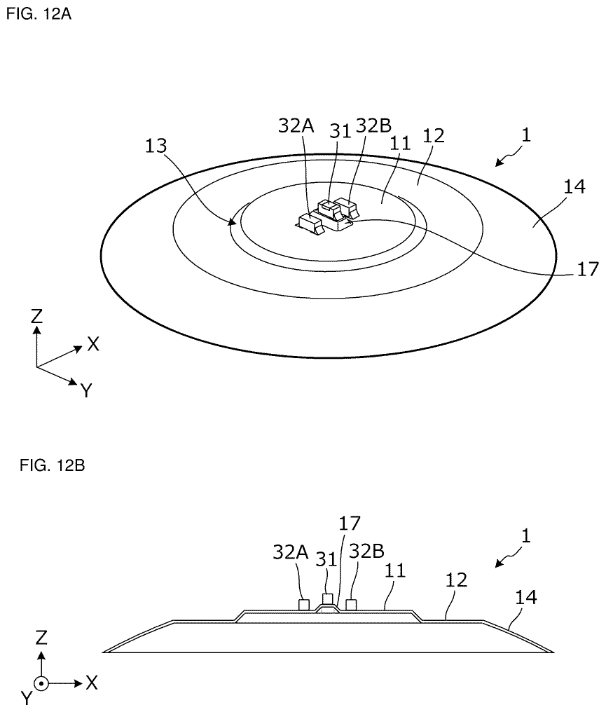

FIG. 12A is a perspective view of a substrate 1 defining a portion of a biological sensor according to the sixth example embodiment. FIG. 12B is a sectional view taken along an X-Z plane at a position passing through the center of the substrate 1. In FIG. 12B, to make illustration of each portion clear, hatching illustration at the sectional position of the substrate 1 is omitted.

A projection-shaped portion 17 is provided on a first portion 11. A light emitter 31 is mounted on an upper surface of the projection-shaped portion 17. Light receivers 32A and 32B are mounted at locations on the first portion 11 where the projection-shaped portion 17 is not provided. Thus, a light-emitting portion of the light emitter 31 is positioned at a location, from the first portion 11, higher than light-receiving portions of the light receivers 32A and 32B.

According to the present example embodiment, the light emitted from the light emitter 31 is hardly directly incident on the light receivers 32A and 32B. That is, wastefully leaked light from the light emitter 31 is not incident on the light receivers 32A and 32B. Thus, a wasted path in light paths from the light emitter 31 to the light receivers 32A and 32B is reduced, and the signal to noise ratio of a biological signal can be increased.

The light receivers 32A and 32B may be mounted on the upper surface of the projection-shaped portion, and the light-receiving portions of the light receivers 32A and 32B may be positioned higher than the light-emitting portion of the light emitter 31.

### Seventh Example Embodiment

In a seventh example embodiment of the present invention, an example of a biological sensor including a coil pattern on a substrate 1 will be described.

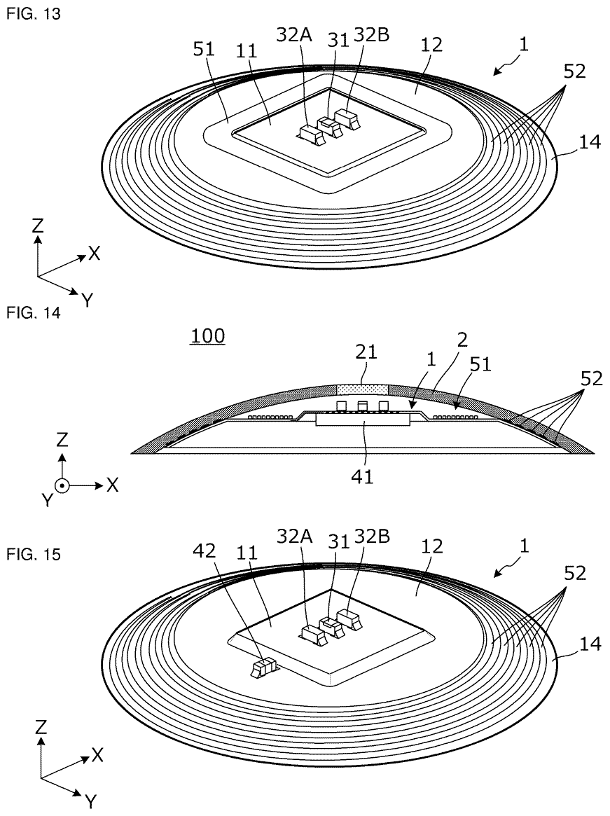

FIG. 13 is a perspective view of the substrate 1 according to the seventh example embodiment. FIG. 14 is a sectional view of a biological sensor 100 including the substrate 1 illustrated in FIG. 13 and a housing 2.

In the present example embodiment, a first coil pattern 51 is provided or mounted on an upper surface of a second portion 12 of the substrate 1. In addition, a second coil pattern 52 is provided or mounted on a curved surface portion 14 of the substrate 1.

The first coil pattern 51 is a square or substantially square conductor pattern including multiple turns, but FIG. 13 illustrates the appearance of the first coil pattern 51 integrally without illustrating each individual pattern of the coil conductor. A first component 41 in FIG. 14 is a square or substantially square plate-shaped magnetic material rather than an IC. The magnetic material acts as a high-permeability magnetic core of the first coil pattern 51, and the first coil pattern 51 is used as, for example, a wireless power-receiving coil.

The second coil pattern 52 is a spiral conductor pattern including multiple turns and is used as, for example, a communication antenna. For example, when in use, one end of the second coil pattern is connected to a communication circuit, and the other end thereof is open.

According to the present example embodiment, the curved surface portion 14 of the substrate 1 can also be effectively used as a portion of an electric circuit.

### Eighth Example Embodiment

In an eighth example embodiment of the present invention, an example of a biological sensor including a first component, a second component, and a second coil pattern on a substrate 1 will be described.

FIG. 15 is a perspective view of the substrate 1 according to the eighth example embodiment. In the present example embodiment, a second component 42 that is a chip component is mounted on an upper surface of a second portion 12 of the substrate 1. In addition, a second coil pattern 52 is provided or mounted on a curved surface portion 14 of the substrate 1. As described above, the second component 42 in the second example embodiment may be provided on the second portion 12 of the substrate 1, and the second coil pattern 52 in the seventh example embodiment may be provided on the curved surface portion 14.

### Ninth Example Embodiment

In a ninth example embodiment of the present invention, an example of a substrate without a curved surface portion and a biological sensor including the substrate will be described.

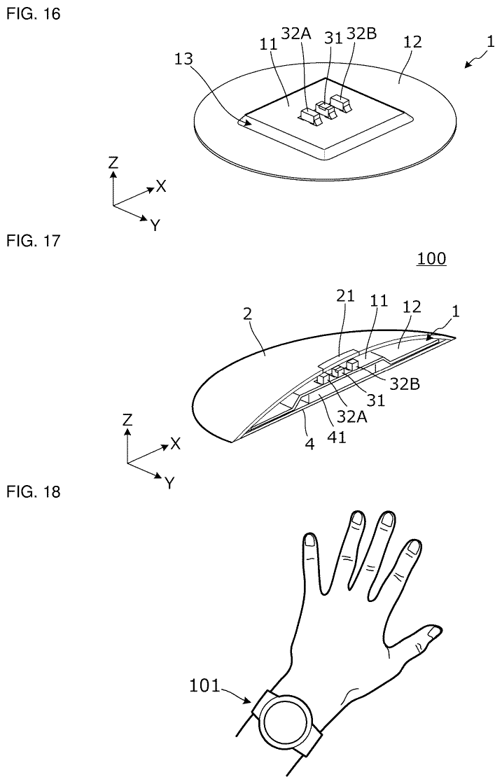

FIG. 16 is a perspective view of a substrate 1 according to the ninth example embodiment. FIG. 17 is a sectional view of a biological sensor 100 including the substrate 1 illustrated in FIG. 16 and a housing 2.

As FIG. 16 illustrates, the substrate 1 includes a first portion 11 and a second portion 12 at mutually different positions in the Z direction and includes a jutting portion 13 between the first portion 11 and the second portion 12 such that the first portion 11 protrudes farther in the first direction than the second portion 12. The first portion 11 of the substrate 1 is provided with a light emitter 31 and light receivers 32A and 32B. The substrate 1 does not include the curved surface portion 14 illustrated in FIG. 1.

As illustrated in FIG. 17, the biological sensor 100 includes the housing 2 including a protuberant curved surface bulging in the Z direction and the substrate 1 inside the housing 2. The housing 2 includes an optical window 21 at a position where the optical window 21 faces the light emitter 31 and the light receivers 32A and 32B. In addition, in the present example embodiment, an opening portion of the housing 2 is covered with a cover 4.

As described above, even without the curved surface portion, by using the protruding portion of the first portion 11 relative to the second portion 12, effective accommodating in the housing 2 including the protuberant curved surface can be achieved.

### Tenth Example Embodiment

In a tenth example embodiment of the present invention, an example of a biological information detecting device including a biological sensor will be described.

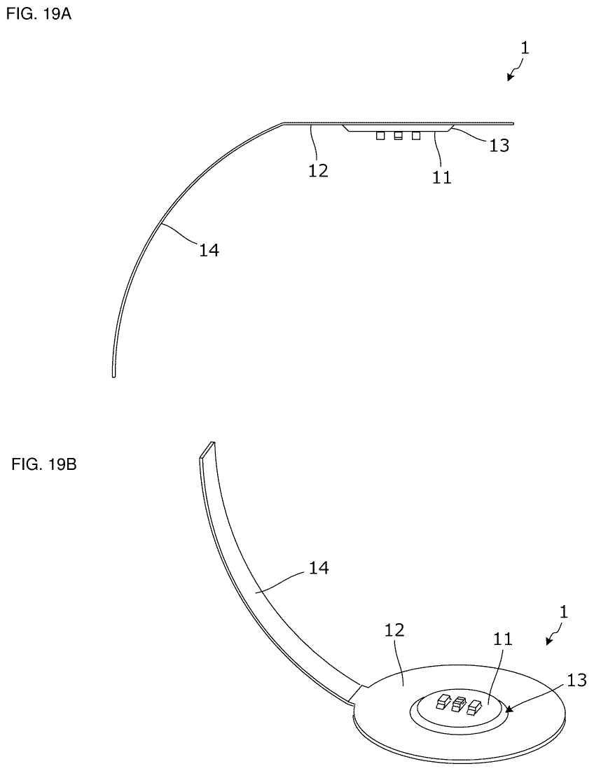

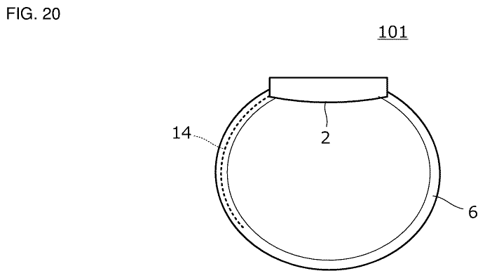

FIG. 18 illustrates a state of a biological information detecting device 101 provided around the left wrist of a human body. FIG. 19A is a side view of a substrate 1 that is a portion of the biological sensor to be accommodated inside the biological information detecting device, and FIG. 19B is a perspective view of the substrate 1. FIG. 20 is a side view of the biological information detecting device 101.

In the state illustrated in FIG. 18, the biological information detecting device 101 is in contact with a living body at a bulging portion of a housing 2. The substrate 1 that is a portion of the biological sensor includes a first portion 11, a second portion 12, a jutting portion 13, and a curved surface portion 14. An antenna is provided at the curved surface portion 14. As illustrated in FIG. 19B, the curved surface portion 14 extends from a portion of the second portion 12. The curved surface portion 14 is accommodated inside a band 6 illustrated in FIG. 20.

In the state illustrated in FIG. 18, the bulging portion of the housing 2 is in contact with a living body, and, for example, pulse frequency and blood oxygen concentration are measured and wirelessly transmitted via the antenna.

According to the present example embodiment, since the curved surface portion 14 of the substrate 1 exists on only a portion of the second portion 12 of the substrate 1 without surrounding the entire periphery of the second portion 12, the curved surface portion 14 can have flexibility.

The present invention is not limited to the above-described example embodiments. Modifications and changes can be performed appropriately by those skilled in the art. The scope of the present invention is indicated by the claims, not the above-described example embodiments. Further, the scope of the present invention includes modifications and changes of the example embodiments within a range equivalent to the claims.

For example, a light receiver may be provided at the center, and a light emitter may be provided at a side portion of the light receiver. In addition, a single light-receiving portion and multiple light emitters may be provided.

In addition, although the examples where the first coil pattern 51 and the second coil pattern 52 illustrated in FIG. 13 and the second coil pattern 52 illustrated in FIG. 15 are disposed on the surface of the substrate 1 are described, such coil patterns may be provided on a lower surface (back surface) of the substrate 1 or inside the substrate 1.

While example embodiments of the present invention have been described above, it is to be understood that variations and modifications will be apparent to those skilled in the art without departing from the scope and spirit of the present invention. The scope of the present invention, therefore, is to be determined solely by the following claims.

What is claimed is:

1. A biological sensor comprising:
a housing including a protuberant curved surface bulging in a first direction; and
a substrate inside the housing; wherein
the biological sensor is to be used with the first direction of the housing directed toward a living body;
the substrate includes a first portion and a second portion at mutually different positions in the first direction, and a jutting portion between the first portion and the second portion such that the first portion protrudes farther in the first direction than the second portion;
a back surface of the first portion and the jutting portion includes a recess; and
a biological detector is provided on a surface of the first portion orthogonal or substantially orthogonal to the first direction.

2. The biological sensor according to claim 1, further comprising a first component including at least a portion mounted on an opposite surface of the first portion that is opposite to a direction of the protruding of the first portion.

3. The biological sensor according to claim 1, further comprising a second component including at least a portion mounted on a surface of the second portion in a direction of the protruding.

4. The biological sensor according to claim 1, wherein the substrate includes a curved surface portion extending along the curved surface of the housing and a multilayer wire portion in or on the curved surface portion.

5. The biological sensor according to claim 1, wherein
the jutting portion of the substrate includes an opening portion;
the housing includes an engagement portion; and
the substrate is combined with the housing into a single body with the opening portion and the engagement portion.

6. The biological sensor according to claim 1, further comprising:
a magnetic material on an opposite surface of the first portion that is opposite to a direction of the protruding of the first portion; and
a first coil pattern on the second portion and turning along a surface of the second portion.

7. The biological sensor according to claim 1, further comprising a second coil pattern turning along the curved surface.

8. The biological sensor according to claim 1, wherein the substrate includes multiple layers of a thermoplastic resin.

9. The biological sensor according to claim 8, wherein the thermoplastic resin is a liquid crystal polymer.

10. The biological sensor according to claim 1, wherein
the first portion includes a projecting body-shaped portion projecting in the first direction; and
the projecting body-shaped portion is in contact with the housing.

11. The biological sensor according to claim 1, wherein
the biological detector includes a light emitter and a light receiver;
the first portion includes a projection-shaped portion projecting in the first direction; and
the projection-shaped portion optically isolates the light emitter and the light receiver from each other.

12. The biological sensor according to claim 11, wherein
the light emitter includes one of an LED, a VCSEL, or a resonator type LED; and
the light receiver includes a photodiode or a phototransistor.

13. A biological information detecting device comprising:
the biological sensor according to claim 1; and
a processor configured or programmed to process detection information of the biological sensor.

14. The biological information detecting device according to claim 13, further comprising a first component including at least a portion mounted on an opposite surface of the first portion that is opposite to a direction of the protruding of the first portion.

15. The biological information detecting device according to claim 13, further comprising a second component including at least a portion mounted on a surface of the second portion in a direction of the protruding.

16. The biological information detecting device according to claim 13, wherein the substrate includes a curved surface portion extending along the curved surface of the housing and a multilayer wire portion in or on the curved surface portion.

17. The biological information detecting device according to claim 13, wherein
the jutting portion of the substrate includes an opening portion;
the housing includes an engagement portion; and
the substrate is combined with the housing into a single body with the opening portion and the engagement portion.

18. The biological information detecting device according to claim 13, further comprising:
a magnetic material on an opposite surface of the first portion that is opposite to a direction of the protruding of the first portion; and
a first coil pattern on the second portion and turning along a surface of the second portion.

19. The biological information detecting device according to claim 13, further comprising a second coil pattern turning along the curved surface.

20. The biological information detecting device according to claim 13, wherein the substrate includes multiple layers of a thermoplastic resin.
